(19) United States
(12) Patent Application Publication
(10) Pub. No.: US 2020/0093958 A1
(43) Pub. Date: Mar. 26, 2020

(54) ALLOGRAFTS CONTAINING AMNIOTIC FLUID AND METHODS THEROF

(71) Applicant: GLOBUS MEDICAL, INC., Audubon, PA (US)

(72) Inventors: Archana Bhat, Phoenixville, PA (US); Breanna Seiber, Philadelphia, PA (US)

(21) Appl. No.: 16/137,626

(22) Filed: Sep. 21, 2018

(51) Int. Cl. A61L 27/36

(52) U.S. Cl. CPC A61L 27/3604; A61L 27/365; A61L 2300/414; A61L 27/3687; A61L 2430/38; A61L 27/3691

(57) ABSTRACT

Allograft biomaterials, implants made therefrom, methods of making the biomaterial and implants, methods of promoting disc, cartilage, tissue, or wound healing in a mammal by administering the biomaterial or implant to the mammal, and kits that include such biomaterials, implants, or components thereof. For example, the allograft may include viable cells, which were native to amniotic fluid that the allograft was derived from.

### TECHNICAL FIELD

The present invention relates generally to cartilage and tissue healing biomaterials, and in particular, allogenic biomaterials containing amniotic fluid. The invention also relates to methods of making the materials and implants, for example, derived from amniotic fluid, and methods of promoting cartilage or wound healing in a mammal by administering the biomaterial or implant to the mammal. The invention further relates to kits that include one or more of the biomaterials, implants, or components thereof.

### BACKGROUND

Cartilage or tissue grafting is a surgical procedure that replaces missing cartilage or tissue and/or repairs cartilage or tissue. Cartilage and tissue generally have the ability to regenerate well but may require a scaffold or other growth enhancers to do so effectively. Grafts may be allograft (e.g., cadaveric origin or live donors), autologous (e.g., tissue harvested from the patient's own body), or synthetic. Cartilage and/or tissue grafts may be resorbed and replaced as the natural cartilage or tissue heals over time.

For cartilage, successful biomaterials may promote chondrogenesis, the process by which cartilage is developed. For other tissues, successful biomaterials may include other suitable pathways or properties to enhance tissue formation and development. Although traditional grafts may exhibit certain advantages, traditional allograft may not exhibit the properties desired, may be difficult to obtain, or may not be in a form suitable for implantation.

### SUMMARY

To meet this and other needs, allograft biomaterials described herein may be configured to promote tissue and/or cartilage healing and repair. The allograft compositions or implants prepared therefrom may be derived, for example, from amniotic fluid. In an exemplary embodiment, the allograft includes viable cells, for example, which were native to the amniotic fluid that the allograft was derived from. The allografts may be particularly suitable for use in cartilage or other tissue healing or when living cells are needing during a surgical procedure.

According to one embodiment, a composition for aiding tissue regeneration includes a mixture of cell pellet containing viable cells obtained from amniotic fluid, decellularized supernatant obtained from the amniotic fluid, and cryoprotectant. The supernatant and cryoprotectant are combined in a ratio of 1:1 and further combined with the cell pellet to achieve a cell count of 350,000-750,000 cells/cc. The composition may be in the form of an injectable or flowable composition and after the composition is frozen and thawed, the composition retains the native viable cells and growth factors in the composition.

According to another embodiment, a method of preparing an implantable composition for aiding tissue regeneration includes obtaining amniotic fluid from a human subject; filtering the amniotic fluid through a macrofilter to form a filtered amniotic fluid; centrifuging the filtered amniotic fluid to obtain pelletized cells and a supernatant; combining the supernatant with a cryoprotectant to form a mixture of the supernatant and the cryoprotectant; and combining the mixture with the pelletized cells to achieve a desired cell count to form the composition. The method may optionally include before combining the supernatant with the cryoprotectant, further centrifuging the supernatant; after centrifuging the supernatant, filtering the supernatant through a 0.45 μm filter, and subsequently, filtering the supernatant through a 0.2 μm sterile filter, thereby resulting in a decellularized and microorganism-free supernatant; after combining the mixture with the pelletized cells to achieve the desired cell count to obtain the composition, dispensing the composition into cryovials and freezing the cryovials at a controlled rate

According to yet another embodiment, a method of promoting disc, tissue, or wound healing in a mammal may include providing an allograft composition, for example, including allograft derived from amniotic fluid; and administering the composition into a target repair site to facilitate repair or regeneration of tissue at the target repair site. The target repair site may be an injury or defect in the spine and the tissue being regenerated may be intervertebral disc.

According to yet another embodiment, a kit includes one or more of the components, compositions, or implants described herein, retrieval kits, trays, syringes, or other components for combining and administering the biomaterial components. The kit may contain compositions of the same or different types. In addition, the kit may include other components known in the art, including, but not limited to, carriers or scaffolds, cages (e.g., titanium and/or polyether ether ketone (PEEK) spacers), allograft spacers, cell culture media, phosphate buffered saline (PBS), a tissue culture substrate, retrieval tools, harvesting tools, implantation tools, or the like.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 depicts steps useful in preparing an amniotic fluid derived composition.

FIG. 2 provides a flowchart of steps in producing the composition according to one embodiment.

FIG. 3 provides a flowchart of steps in producing the composition according to another embodiment.

### DETAILED DESCRIPTION

The present invention relates generally to allograft biomaterial compositions and implants made therefrom that may be used in a variety of surgical procedures. The invention also relates to methods of making the compositions and implants, and methods of promoting cartilage, tissue, or wound healing in a mammal by administering the biomaterial or implant to the mammal. The invention further relates to kits that include one or more of the biomaterials, implants, retrieval kits, tools and trays for administering the composition, and other components thereof.

Additional aspects, advantages and/or other features of example embodiments of the invention will become apparent in view of the following detailed description. It should be apparent to those skilled in the art that the described embodiments provided herein are merely exemplary and illustrative and not limiting. Numerous embodiments of modifications thereof are contemplated as falling within the scope of this disclosure and equivalents thereto.

In describing example embodiments, specific terminology is employed for the sake of clarity. However, the embodiments are not intended to be limited to this specific terminology. Unless otherwise noted, technical terms are used according to conventional usage.

As used herein, “a” or “an” may mean one or more. As used herein “another” may mean at least a second or more. As used herein, unless otherwise required by context, singular terms include pluralities and plural terms include the singular.

As used herein and in the claims, the terms “comprising” and “including” are inclusive or open-ended and do not exclude additional unrecited elements, compositional components, or method steps. Accordingly, the terms “comprising” and “including” encompass the more restrictive terms “consisting essentially of” and “consisting of.”

Unless specified otherwise, all values provided herein include up to and including the endpoints given, and the values of the constituents or components of the compositions are expressed in weight percent or % by weight of each ingredient in the composition.

Each compound or name used herein may be discussed interchangeably with respect to its chemical formula, chemical name, abbreviation, acronym, etc. For example, DMSO may be used interchangeably with dimethyl sulfoxide.

Embodiments described herein may be generally directed to allograft biomaterial compositions, implants made therefrom, methods of making the same, and methods of using the same to promote healing of tissue and/or cartilage repair. Although compositions, biomaterials or implants may be discussed separately, it will be appreciated by one of ordinary skill in the art that the compositions or biomaterials described may be used in and of itself or may be used to create implants of different shapes, sizes, and orientations for a number of different clinical outcomes. Thus, the discussion of biomaterials or compositions may apply equally to the discussion on implants and vice versa.

Low back pain as a result of degenerative disc disease (DDD) affects a large patient population aged 45 years and older. Traditional concepts for treatment range from pain reliefs from steroidal injections in mild/moderate DDD to limiting motion at the affected segment in severe stages of DDD. Recent advancements in treating DDD have involved development of stem cell therapies, where the cells may be isolated from bone marrow, disc material etc. However, the avascular, hypoxic nature of the disc together with the limit of nutritional transport across the mineralized end plates, make the disc a difficult environment for the cells to survive. Hence, a need to find the optimized solution to treat this condition still remains imperative.

The amniotic fluid possesses anti-inflammatory and regenerative properties that make it an attractive material for use in treating discogenic pain. According to one embodiment, the allograft compositions or implants prepared therefrom may be derived, for example, from amniotic fluid. In an exemplary embodiment, the allograft includes viable cells, such as stem cells, and/or growth factors. In other words, viable cells present in the allograft may be alive and capable of growth. The viable cells and/or growth factors may be native to the amniotic fluid. In other words, native cells, growth factors, and/or other components of the allograft may include the original cells and tissues present in the amniotic fluid when obtained from the donor. The native cells do not include exogenous, cultured, or expanded cells, although it is envisioned that such additional cells may be added to the allograft material, if desired. Similarly, the allograft may include only native tissues and components present in the amniotic fluid when obtained from the donor or may be combined with other tissues, natural materials, synthetics, or other components, for example, suitable to promote tissue regeneration and improve the handling and delivery of the product to the target site. In an exemplary embodiment, the composition is an injectable composition containing stem cells and growth factors from the amniotic fluid configured for treating chronic lower back pain.

When used for cartilage or disc repair, the allograft biomaterial compositions may be chondrogenic. Chondrification or chondrogenesis is the process in which cartilage is formed. The cartilage may be formed from condensed mesenchyme tissue, which differentiates into chondrocytes, and secretes the molecules that form extracellular matrix for cartilage repair. Once damaged, cartilage may have limited natural repair capabilities. Because chondrocytes are bound in lacunae, they may not be able to naturally migrate to damaged areas. Thus, the allograft biomaterial compositions may contain chondrocytes, chondrogenic precursors, or other properties suitable for promoting chondrogenesis, thereby ultimately promoting cartilage or disc repair.

When used for other tissue healing or regeneration, the allograft biomaterial compositions may be configured to otherwise promote tissue healing. Tissue repair may be characterized by increased cell proliferation, capillary budding, and the synthesis of extracellular matrix (ECM) to fill in the damaged tissue. Thus, the allograft biomaterial compositions may contain cells, precursors, or other properties suitable for promoting tissue healing and repair. For example, other tissues may include epithelial tissue, connective tissue, muscle tissue, or nerve tissue.

The composition may also be “biocompatible” as that term refers to the ability (e.g., of a composition or material) to perform with an appropriate host response in a specific application, or at least to perform without having a toxic or otherwise deleterious effect on a biological system of the host, locally or systemically. The biomaterial or a portion thereof may be “biologically degradable” in that the material may be degraded by cellular absorption and/or hydrolytic degradation in a patient's body.

According to one embodiment, the allograft biomaterial compositions may be configured to facilitate repair or regeneration of tissue, for example, cartilage or other tissue. In particular, the allograft biomaterial compositions may facilitate repair or regeneration of tissue at a target repair site. The target repair site can be, for example, a void, gap, or other defect, or a surgeon created opening in disc, cartilage, between bones, or other structure or tissue location in a body of a patient. The allograft biomaterial compositions may be configured to facilitate cartilage or other tissue growth at a target repair site. The allograft biomaterial compositions may be configured to be directly implanted or otherwise disposed at and in contact with the target repair site. The patient and target repair site may be in a human, mammal, or other organism.

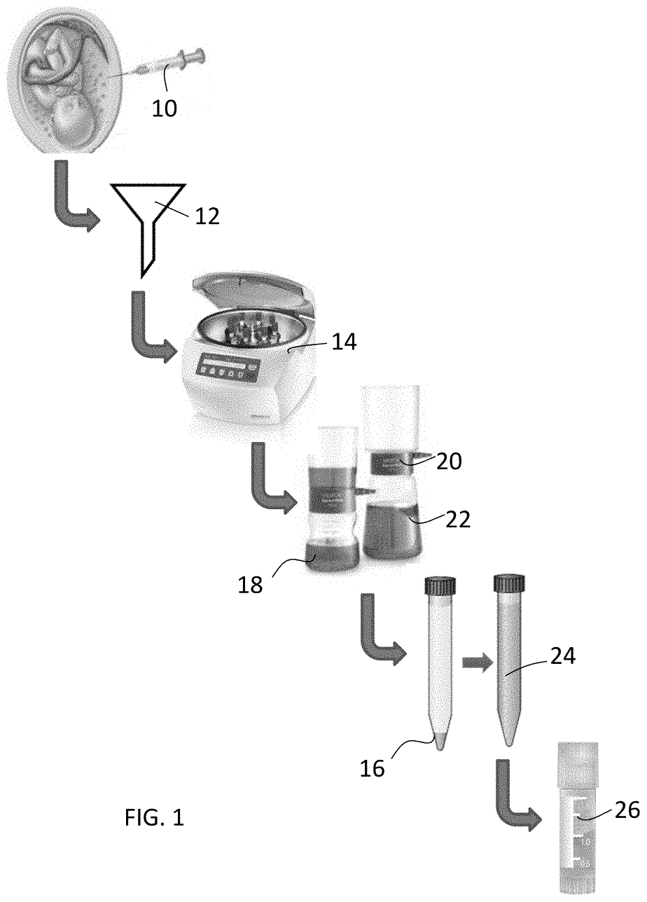

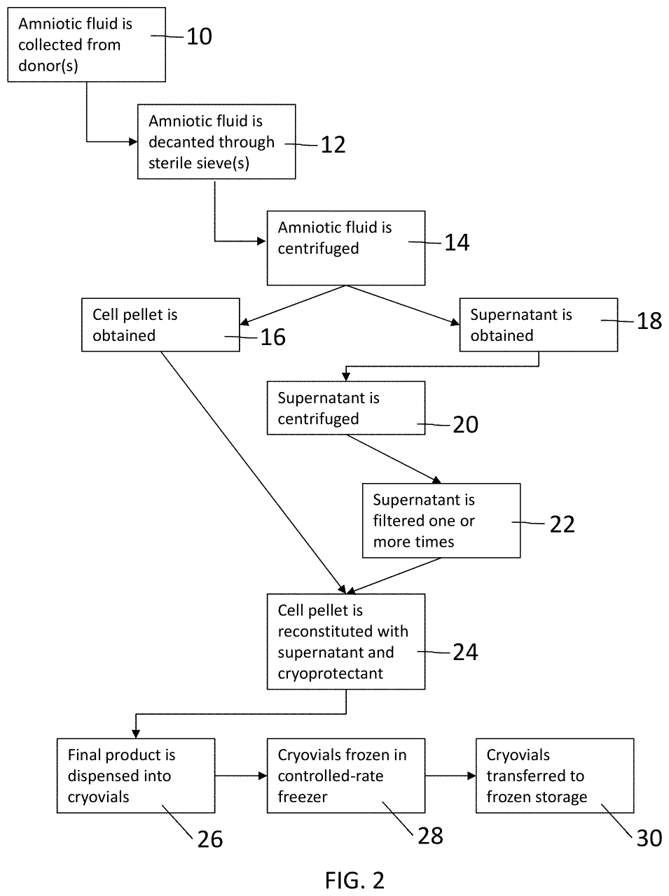

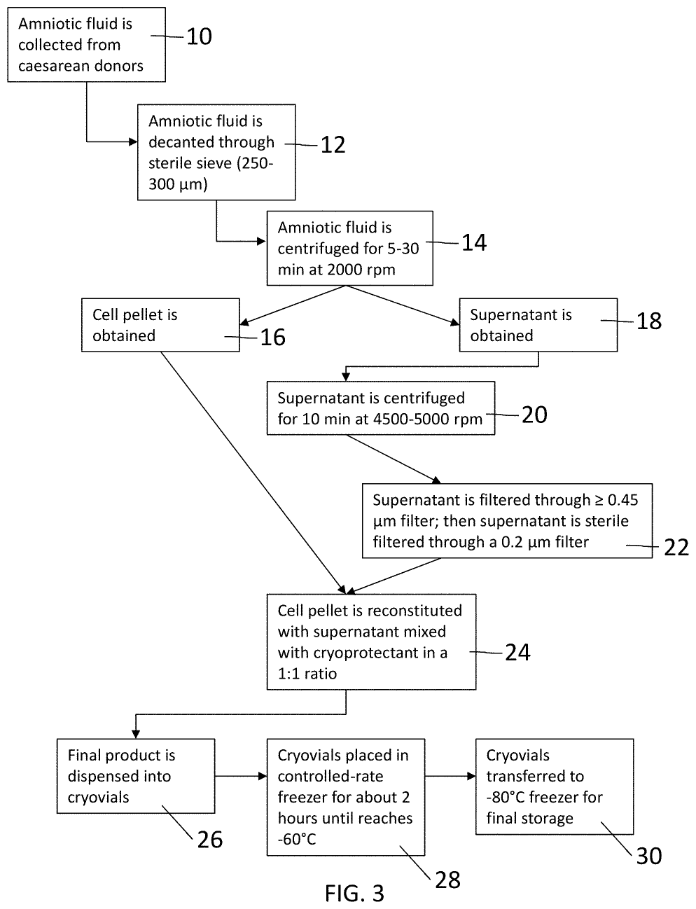

According to one embodiment, a composition for aiding tissue regeneration includes allograft derived from amniotic fluid. As best seen in FIGS. 1-3, in step 10, amniotic fluid is collected from one or more donors. The amniotic fluid may be collected, for example, during a caesarean procedure or any other suitable procedure. The amniotic fluid is preferably processed in a short period of time to maintain the viability of the native cells, growth factors, and other native components. For example, the amniotic fluid is preferably processed within 72 hours of recovery, 48 hours of recovery, or as soon as practical.

Turning to step 12, the amniotic fluid may be filtered. For example, the fluid may be decanted through a sterile sieve. This filtration step 12 may provide for macrofiltration of the amniotic fluid to remove any large particles, debris, or other unwanted remnants in the amniotic fluid. For example, the filtration step 12 may be conducted with a sieve having a pore size of about 50-500 μm, about 50-300 m, about 200-400 μm, about 200-250 μm, or about 250-300 μm.

Moving next to step 14, the macro-filtered amniotic fluid may be centrifuged or otherwise separated. Centrifugation is a separation process which uses the action of centrifugal force to promote accelerated settling of particles in a solid-liquid mixture. In particular, the amniotic fluid may be centrifuged sufficiently to pelletize the cells and other solid elements (e.g., viable stem cells, growth factors). The amniotic fluid may be centrifuged for about 1-60 minutes, or about 5-30 minutes at, for example, about 1000-3000 rpm, about 1500-2500 rpm, or about 2000 rpm to pellet the cells. Following centrifugation, the cell pellet is obtained in step 16 and the liquid supernatant is obtained in step 18. The cell pellet 16 may contain all or mostly all of the viable cells and other solid components of the original amniotic fluid. The supernatant 18 may be mostly or entirely decellularized and contain all of the liquid portion remaining from the amniotic fluid. The supernatant from step 18 may be further processed.

In step 20, the resulting supernatant 18 may be further centrifuged. For example, the supernatant may undergo an additional centrifugation cycle for a minimum of 10 minutes (e.g., about 10-60 minutes) at about 1000-6000 rpm, about 3000-5000 rpm, or about 4500-5000 rpm.

In step 22, the resulting supernatant 20 may be filtered one or more times. The filtration step 22 may provide for microfiltration of the amniotic fluid. The supernatant may be filtered through a sieve with a greater than or equal to 0.45 μm filter for the first-pass macro filtration. Microfiltration with a membrane filter with a 0.45 μm pore size may be used to remove bacteria and other microorganisms from the sample. Subsequently, the fluid may be sterile filtered using a 0.1 to 0.45 μm filter, or a 0.2 μm filter, for example. A 0.2 μm filter may allow for final sterilization and final filtration of the fluid.

As shown in step 24, the microfiltered and sterile filtered supernatant 22 may be recombined with the cell pellet 16. In particular, the cells may be reconstituted with the sterile filtered supernatant mixed with a cryoprotectant. The cryoprotectant may include a low molecular weight dextran, dextrose, dimethyl sulfoxide, minimum essential medium, glycerol, polyethylene glycol (PEG), any combination thereof, or other suitable cryoprotectant. In an exemplary embodiment, the cryoprotectant is 10% low molecular weight dextran in a 5% dextrose solution. In a preferred embodiment, the supernatant and cryoprotectant are combined in about a 1:1 ratio. The supernatant and cryoprotectant may also be combined in other suitable ratios, ranging from 100% cryoprotectant to 100% supernatant. The cells may be reconstituted with the supernatant/cryoprotectant mixture to achieve a cell concentration of about 100,000-1,000,000 cells/cc, about 200,000-800,000 cells/cc, about 350,000-750,000 cells/cc, or about 750,000 cells/cc. The cell counts may be based on viable cells, such as stem cells, within the composition. The cell pellet may be mixed with the supernatant/cryoprotectant mixture using any suitable techniques to re-suspend the cells, growth factors, and particulates within the fluid.

As shown in step 26, the final product may be dispensed into cryovials, for example, containing about 1-5 cc each. The cryovials may be placed into a controlled-rate freezer for approximately 2 hours, for example. The freezer program may be used to gradually bring the temperature to about −60° C. When the freezer program is complete, the product may be transferred to a −80° C. freezer for final storage shown in step 30. It is envisioned that any other suitable freezing rates and temperatures may be used.

In one embodiment, the amniotic fluid may be decanted through a sterile graduated sieve (250-300 μm) and centrifuged for 5-30 minutes at 2000 rpm to pellet the cells. The supernatant may undergo an additional centrifugation cycle for a minimum of 10 minutes at 4,500-5,000 rpm. The supernatant may be filtered through a ≥0.45 μm filter for the first-pass macro-filtration and sterile filtered using a 0.2 μm filter. The cells may then be reconstituted in sterile filtered supernatant mixed with cryoprotectant (10% LMD [low molecular weight dextran] in 5% dextrose) in a 1:1 ratio. The cells may be reconstituted to achieve a cell concentration of 350,000-750,000 cells/cc. The final product may be dispensed into cryovials (1-5 cc each) and placed into a controlled-rate freezer for approximately 2 hours. The freezer program gradually brings the temperature to −60° C. When the freezer program is complete, the product may be transferred to a −80° C. freezer for final storage.

According to an alternative embodiment, the amniotic fluid may be filtered using a 50 micron filter and centrifuged to pellet the cells. The cells may then be reconstituted in alpha minimum essential medium (alpha MEM) mixed with 5% dextrose or Dulbecco's minimum essential medium (DMEM) mixed with 5% dextrose solution. The cell pellet may also be reconstituted in alpha MEM or DMEM with 10% di-methyl sulphoxide (DMSO). The cells may be reconstituted to achieve a cell concentration of about 350,000 cells/cc-750,000 cells/cc. 1-5 cc of product may be dispensed into cryovials and stored at −80C.

According to yet another embodiment, the amniotic fluid may be filtered using a 50 micron filter. The filtrate may be mixed with 10% DMSO that serves as a cryopreserving agent. 1-5 cc of the product may be dispensed into the cryovials and stored at −80C.

According to yet another alternative embodiment, the amniotic fluid may be filtered using a 50 micron filter and centrifuged to pellet the cells. The cells may then be reconstituted back in the supernatant mixed with 10% DMSO. Reconstitution volume may be tailored to achieve a cell concentration of about 350,000 cells/cc-750,000 cells/cc. 1-5 cc of the product may be dispensed into the cryovials and stored at −80C.

Although it is envisioned that the amniotic fluid-derived allograft may be used alone, it is also envisioned that the allograft may combined with other components. For example, one or more carriers, scaffold materials, or processing additives may be used with the allograft composition. Suitable carriers, scaffolds, or additives may include, but are not limited to, minimum essential medium (e.g., alpha MEM or DMEM), demineralized bone matrix (DBM) or other bone-derived components, ceramics including bioactive glasses or tricalcium phosphates, collagen including soluble and insoluble collagen, bone morphogenetic proteins (BMPs), phospholipids, carboxylmethylcellulose (CMC), glycerin, glycerol, polyethylene glycol (PEG), hydrogels, poloxamers, polylactic acid (PLA), polylactic-co-glycolic acid (PLGA), other copolymers of the same family, and combinations thereof.

Additionally, biological agents may be added to the biomaterial or implant, such as additional growth factors such as platelet derived growth factor (PDGF), vascular endothelial growth factor (VEGF), insulin derived growth factor (IDGF), a keratinocyte derived growth factor (KDGF), or a fibroblast derived growth factor (FDGF), stem cells, and platelet rich plasma (PRP), to name a few. If desired, one or more active pharmaceutical ingredients or medicaments may be incorporated into the biomaterial as well. Biological agents may be added in any suitable pharmaceutically acceptable and effective amounts known in the art.

When ready to be implanted in a patient, the frozen mixture may be thawed prior to use. In particular, at point of care, the vial may be thawed and the product may be in a fluid or flowable form. The amniotic product may be injected into the affected disc, for example, using an 18-22-gauge needle. Amniotic fluid may have an average of 304 cytokines along with a minor population of cells that display mesenchymal stem cell (MSC) phenotype. Due to minimal processing, the living cells (e.g., stem cells) and/or growth factors remain viable in the allograft. The combination of growth factors and cells may help augment the regeneration process, and the resulting allografts may be particularly suitable for intervertebral disc repair. The human allograft, derived from amniotic fluid, may be used, for example, to treat degenerative disc disease (DDD) and may be a suitable replacement for spinal fusion surgery. Additionally, the anti-inflammatory properties of the composition could help reduce DDD inflicted inflammation in the disc.

The allograft biomaterials described herein and/or implants formed therefrom are intended to be applied at a tissue or cartilage repair site, e.g., one resulting from injury or defect. The implant can be utilized in a wide variety of orthopedic, periodontal, neurosurgical, oral and maxillofacial surgical procedures. In particular, the biomaterials may be suitable for repairs of the vertebral column including spinal fusion and internal fixation; tumor surgery, e.g., deficit filling; discectomy; laminectomy; scoliosis, lordosis and kyphosis treatments. Possible clinical applications may include e.g., the treatment of spinal disc degeneration or disease, traumatic, pathologic, or stress fractures, congenital defects or fractures, or operative defects near any bone or between bones of the body.

The compositions and implants may be configured for use at various target repair sites within a body of a patient to facilitate cartilage and/or tissue growth therein. In some embodiments, the composition is configured for use at a target repair site in the patient's spine. For example, the composition can facilitate chondrogenic repair of the intervertebral disc between adjacent vertebrae. In a spinal fusion procedure, the composition may be used in conjunction with one or more mechanical supports (e.g., a cage or frame, spacer, plate, a plurality of screws and/or rods, or the like). Although the spine is described, the composition can be configured to be implanted into or at a target repair site in or at a different cartilage, tissue or other structures of the patient's body.

The term “treating” and the phrases “treatment of a disease” and “treatment of a condition” refer to executing a protocol that may include the use of the compositions, devices and methods herein and/or administering one or more biomaterials to a patient (human, normal or otherwise, or other mammal), in an effort to alleviate signs or symptoms of the disease or condition. Alleviation can occur prior to signs or symptoms of the disease or condition appearing, as well as after their appearance. Thus, “treating” or “treatment” includes “preventing” or “prevention” of disease or undesirable condition. In addition, “treating” or “treatment” does not require complete alleviation of signs or symptoms and does not require a cure to the ailment.

Further example embodiments are directed to kits that include components for making the present biomaterials and implants, including for example, carriers or scaffolds, cages (e.g., titanium and/or polyether ether ketone (PEEK) spacers), allograft spacers, demineralized bone materials, cell culture media, phosphate buffered saline (PBS), a tissue culture substrate such as a flask, trypsin, or mixtures, harvesting tools, retrieval tools, or the like. Additional components, instructions and/or other apparatus may also be included.

Although the invention has been described in example embodiments, those skilled in the art will appreciate that various modifications may be made without departing from the spirit and scope of the invention. It is therefore to be understood that the inventions herein may be practiced other than as specifically described. Thus, the present embodiments should be considered in all respects as illustrative and not restrictive. Accordingly, it is intended that such changes and modifications fall within the scope of the present invention as defined by the claims appended hereto.

What is claimed is:

1. A method of preparing an implantable composition for aiding tissue regeneration, the method comprising:
obtaining amniotic fluid from a human subject;
filtering the amniotic fluid through a macrofilter to form a filtered amniotic fluid;
centrifuging the filtered amniotic fluid to obtain pelletized cells and a supernatant;
combining the supernatant with a cryoprotectant to form a mixture of the supernatant and the cryoprotectant; and
combining the mixture with the pelletized cells to achieve a desired cell count to form the composition.

2. The method of claim 1, wherein the amniotic fluid is macrofiltered through a sterile sieve having a pore size of 250-300 μm.

3. The method of claim 1, wherein the cryoprotectant includes dextran, dextrose, or a combination thereof.

4. The method of claim 3, wherein the cryoprotectant includes 10% low molecular weight dextran in 5% dextrose.

5. The method of claim 1, wherein the supernatant is combined with the cryoprotectant in a ratio of 1:1.

6. The method of claim 1, wherein the pelletized cells contain native stem cells and growth factors.

7. The method of claim 1, wherein the desired cell count is 350,000-750,000 cells/cc.

8. The method of claim 1, further comprising before combining the supernatant with the cryoprotectant, further centrifuging the supernatant.

9. The method of claim 8, further comprising after centrifuging the supernatant, filtering the supernatant through a 0.45 μm filter, and subsequently, filtering the supernatant through a 0.2 μm sterile filter, thereby resulting in a decellularized and microorganism-free supernatant.

10. The method of claim 1, further comprising after combining the mixture with the pelletized cells to achieve the desired cell count to obtain the composition, dispensing the composition into cryovials and freezing the cryovials at a controlled rate.

11. The method of claim 10, wherein the cryovials are frozen and maintained at a temperature of from −60° C. to −80° C.

12. The method of claim 1, further comprising thawing the composition before use, wherein the composition is an injectable composition.

13. A composition for aiding tissue regeneration, the composition comprising:
a mixture of cell pellet containing viable cells obtained from amniotic fluid, decellularized supernatant obtained from the amniotic fluid, and cryoprotectant, wherein the supernatant and cryoprotectant are combined in a ratio of 1:1 and further combined with the cell pellet to achieve a cell count of 350,000-750,000 cells/cc.

14. The composition of claim 13, wherein the composition is an injectable composition.

15. The composition of claim 13, wherein when the composition is frozen and subsequently thawed, the composition retains the viable cells.

16. The composition of claim 13, wherein the cryoprotectant includes dextran, dextrose, or a combination thereof.

17. The composition of claim 13, wherein when the amniotic fluid is macrofiltered and centrifuged to form the cell pellet and supernatant, and the supernatant is further centrifuged and filtered.

18. The composition of claim 13, wherein the supernatant is further centrifuged and filtered through a 0.45 μm filter, and subsequently, through a 0.2 μm sterile filter, thereby resulting in the decellularized and microorganism-free supernatant.

19. A method of promoting bone or treating degenerated disc in a mammal, the method comprising:
providing the composition of claim 13; and
administering the composition into a target repair site to facilitate repair or regeneration of tissue at the target repair site.

20. The method of claim 19, wherein the target repair site is an injury or defect in the spine and the tissue being regenerated is disc.
